(19) United States
(12) Patent Application Publication
(10) Pub. No.: US 2007/0014855 A1
(43) Pub. Date: Jan. 18, 2007

(54) STABLE DESLORATADINE COMPOSITIONS

(76) Inventors: Gawande S. Rahul, Nagpur (IN); Praveen Kumar Subbappa, Coorg (IN); Ravinder Kodipyaka, Sirpur-Kaghaznagar (IN); Vijay Dinanathji Nasare, Nagpur Katol (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/456,894

(22) Filed: Jul. 12, 2006

Related U.S. Application Data
(60) Provisional application No. 60/736,020, filed on Nov. 10, 2005.

(30) Foreign Application Priority Data
Jul. 12, 2005 (IN) 927/CHE/2005

(51) Int. Cl. A61K 9/20

(52) U.S. Cl. 424/464

(57) ABSTRACT

Stable pharmaceutical compositions of desloratadine or pharmaceutically acceptable salts in combination with one or more therapeutically active compounds.

The present invention relates to stabilized desloratadine (also called descarbonylethoxyloratadine) compositions. More particularly, the present invention relates to stable pharmaceutical compositions of desloratadine or pharmaceutically acceptable salts, solvates, enantiomers or mixtures thereof in combination with one or more therapeutically active compounds or their pharmaceutically acceptable salts, solvates, single isomer, enantiomers or mixtures and processes for preparing the same.

Desloratadine, having the chemical name (8-chloro-6,11-dihydro-11-(4-piperidylidene)-5H-benzo[5,6]-cyclohepta[1,2-b]pyridine, is a metabolic derivative of loratadine and is useful as a non-sedating antihistamine. Desloratadine is a white to off-white powder that is slightly soluble in water, but very soluble in ethanol and propylene glycol. Pseudoephedrine, chemically (+)-(1S,2S)-2-methylamino-1-phenylpropan-1-ol, is an orally effective sympathomimetic nasal decongestant. A commercially available combination of desloratadine with pseudoephedrine sulphate is CLARINEX™ D 24 HOUR tablets (5 mg/240 mg) is double layered, wherein desloratadine is in an immediate release layer and pseudoephedrine in an extended release layer, and is manufactured by Schering Corporation. Desloratadine is described by the structural Formula I.

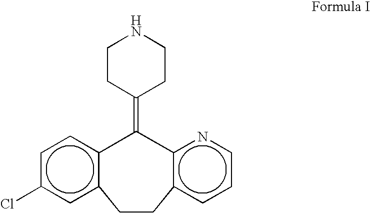

Desloratadine and its compositions are prone to oxidation and decomposition by acidic excipients to form impurities such as deschlorodesloratadine, dehydrodesloratadine and N-formyldesloratadine, more specifically N-formyldesloratadine.

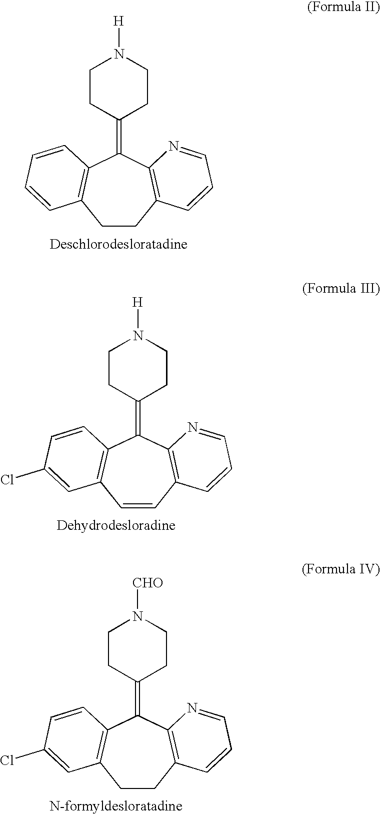

Journal of Pharmaceutical Sciences, 1998, 87(1): 31-39 published an article by Wirth, et. al., “Maillard reaction of lactose and fluoxetine hydrochloride, a secondary amine” which concludes that not only primary amine but secondary amines also undergo the Maillard reaction with lactose under pharmaceutically relevant conditions. The author identified N-formyl fluoxetine as a major product of the Maillard reaction between fluoxetine, a secondary amine, and lactose.

U.S. Pat. No. 6,100,274 describes desloratadine and its pharmaceutical compositions comprising desloratadine and a desloratadine-protective amount of a pharmaceutically acceptable calcium phosphate salt.

U.S. Pat. No. 6,709,676 discloses a bilayered solid composition comprising desloratadine and pseudoephedrine.

U.S. Pat. No. 6,979,463 discloses a film coated extended release composition of pseudoephedrine sulfate in a core, and a film coating comprising desloratadine for immediate release, the core and desloratadine coat being separated by an intermediate seal coat. In the event of incompatibility between desloratadine and acidic excipients, including hydroxypropylmethyl cellulose, the inventors used an intermediate seal coat comprising a water swellable, film forming neutral or cationic copolymer ester (Eudragit®) that is compatible with desloratadine. The invented composition showed total desloratadine degradation products less than or equal to 1 to 1.5 weight percent on storage for at least 24 months at 25° C. and about 60% RH.

U.S. Patent Application Publication No. 2002/0123504 A1 describes pharmaceutical compositions of desloratadine that do not contain lactose. Also disclosed is a method for preventing contact between desloratadine and reactive formulation components by applying an inert coating to desloratadine granules.

International Patent Application Publication No. WO 2005/065047 describes desloratadine compositions that include a stabilizer selected from an antioxidant, an organic compound providing an alkaline pH, and an alkali metal salt.

The present invention provides a solution for a long felt need in the stabilization of compositions of desloratadine in combination with one or more therapeutically active compounds to prevent the decomposition and discoloration and thereby giving a stable composition with any individual impurity less than about 1% when stored at 25° C. and a relative humidity of 60% or at accelerated conditions such as 40° C. and a relative humidity of 75%.

Hence, a pharmaceutical composition of desloratadine using stabilizers that are effective in small quantities compared to the compositions described in literature as described above, or seperation of desloratadine layer from other therapeutica active ingredients by use of a inert barrier layer, in combination with one or more therapeutically active compounds would be a significant improvement in the formulation and its use in the therapy of allergic rhinitis and other histamine-induced disorders.

### SUMMARY OF THE INVENTION

The present invention relates to stabilized desloratadine compositions. More particularly, the present invention relates to stable pharmaceutical compositions of desloratadine or pharmaceutically acceptable salts, solvates, enantiomers or mixtures thereof in combination with one or more therapeutically active compounds or their pharmaceutically acceptable salts, solvates, single isomer, enantiomers or mixtures and processes for preparing the same.

The present invention relates to pharmaceutical compositions of desloratadine comprising desloratadine, a desloratadine stabilizer such as metallic oxides, amines or amino acids or mixtures thereof or formation of a stable complex of an anti-allergic effective amount of desloratadine with porous copolymers of methacrylic acid crosslinked with divinylbenzene, which is an ion exchange resin, or by providing an inert barrier layer of hydrophilic polymer that separates desloratadine from other therapeutically active compounds.

In another aspect the invention provides a pharmaceutical composition of desloratadine in combination with one or more therapeutically active compounds with improved stability of the composition having less than 1% of N-formyldesloratadine impurity.

A pharmaceutical composition according to the present invention may be obtained by stabilizing desloratadine or pharmaceutically acceptable salts, solvates, enantiomers or mixtures thereof with a stabilizer or by providing an inert barrier layer of hydrophilic polymer that separates desloratadine from other therapeutically active compounds.

This invention thus provides composition comprising an immediate release first layer of an anti-allergic effective amount of desloratadine and a desloratadine-protective amount of a pharmaceutically acceptable carrier medium comprising substances, which are basic in nature, or a stable complex of desloratadine with the porous copolymers of methacrylic acid crosslinked with divinylbenzene or by providing an inert barrier layer of hydrophilic polymer that separates desloratadine from other therapeutically active compounds. The other layers comprise an effective amount of one or more therapeutically active compounds or their pharmaceutically acceptable salts, solvates, enantiomers or mixtures.

### DETAILED DESCRIPTION OF THE INVENTION

The present invention relates to stabilized desloratadine compositions. More particularly, the present invention relates to stable pharmaceutical compositions of desloratadine or pharmaceutically acceptable salts, solvates, enantiomers or mixtures thereof in combination with one or more therapeutically active compounds or their pharmaceutically acceptable salts, solvates, single isomer, enantiomers or mixtures and processes for preparing the same.

The present invention relates to pharmaceutical compositions of desloratadine comprising desloratadine, a desloratadine stabilizer such as metallic oxides, amines or amino acids or mixtures thereof or formation of a stable complex of an anti-allergic effective amount of desloratadine with porous copolymers of methacrylic acid crosslinked with divinylbenzene, which is an ion exchange resin, or by providing an inert barrier layer of hydrophilic polymer that separates desloratadine from other therapeutically active compounds.

The present invention provides a pharmaceutical composition of desloratadine having an N-formyldesloratadine impurity less than about 1%, using an alkaline substance as a stabilizer, or forming a stable complex of desloratadine with porous copolymers of methacrylic acid crosslinked with divinylbenzene, or by providing an inert barrier layer of hydrophilic polymer that separates desloratadine from other therapeutically active compounds.

Stability of pharmaceutical compositions may be defined as the capability of a particular dosage form, in specified packaging, to maintain its physical, chemical, microbiological, therapeutic and toxicological specifications. There are standard tests for determining stability of pharmaceutical products, involving storage under controlled conditions of temperature and relative humidity (“RH”), with periodic analysis of samples of the stored material. A widely performed “accelerated stability test” uses storage conditions of 40° C. and 75% RH.

Stability of pharmaceutical compositions may be affected by several factors, including the stability of the active pharmaceutical ingredient (“API”), API-excipient incompatibilities, and mode of packaging. Factors such as oxidation, moisture, heat and light may initiate and/or accelerate a chemical interaction, thereby degrading the API in a composition.

Desloratadine compositions degrade to form impurities including the compounds deschlorodesloratadine, dehydrodesloratadine and N-formyldesloratadine, more specifically N-formyldesloratadine, and the present invention provides a stabilized composition containing less than 1% N-formyl desloratadine. All impurity concentrations stated herein are weight percentages of the initial desloratadine content, and are not to be interpreted as percentages of the entire composition.

Surprisingly, the inventors have found that in spite of incompatibility reported between desloratadine and acidic excipients such as stearic acid, povidone, crospovidone, lactose, ethyl cellulose, and hydroxypropyl methylcellulose, desloratadine, pharmaceutical compositions of the present invention remained stable at about 40° C. and about 75% RH for 3 months, showing 0.3% w/w N-formyldesloratadine impurity and 1% w/w total degradation products, when an inert barrier layer of hydroxypropyl methylcellulose was provided between layer comprising desloratadine and the hydrophilic core matrix comprising pseudoephedrine sulfate.

Desloratadine compositions in combination with one or more therapeutically active compounds with improved stability are achieved by using an alkaline substance such as a metal oxide, or an amine, or an amino acid, or combinations thereof, or forming a stable complex of desloratadine with porous copolymers of methacrylic acid crosslinked with divinylbenzene, and we also have determined that providing an inert barrtier layer onto core containing one or more therapeutically active compounds, e.g., pseudoephedrine sulphate, with a cellulosic polymer, e.g., a hydroxypropyl methylcellulose layer, and then layering a desloratadine composition over the core has resulted in stable compositions.

An aspect of the present invention relates to stable pharmaceutical compositions of desloratadine wherein desloratadine is present in an intimate admixture with alkaline substances, or in a stable complex with porous copolymers of methacrylic acid crosslinked with divinylbenzene and other pharmaceutically acceptable excipients, including, but not limited to, blended, granulated or compressed dosage forms, that provides stabilization to the desloratadine in the composition.

In an embodiment the present invention provides stable pharmaceutical compositions comprising desloratadine or pharmaceutically acceptable salts, solvates, enantiomers or mixtures thereof, blended or granulated with an alkaline stabilizer or in an ion exchange resin complex, and other pharmaceutically acceptable excipients.

The present invention, in one of the embodiments, provides for stabilization of desloratadine using amines or amino acids as stabilizers, and a pharmaceutical composition comprising such stabilized desloratadine and pseudoephedrine as another active ingredient formulated as bilayer tablets.

The present invention, in another embodiment, provides for stabilization of desloratadine by formation of resinate complexes with ion exchange resins, and a pharmaceutical composition comprising a desloratadine resinate complex and pseudoephedrine as another active ingredient, formulated as bilayer tablets.

One of the embodiments of the present invention provides for application of an inert barrier layer of hydrophilic polymer, such as hydroxypropyl methylcellulose, between the desloratadine layer and the hydrophilic core matrix comprising pseudoephedrine sulfate.

Another embodiment of the invention comprises:
a) a first layer presented as a core comprising one or more therapeutically active compounds with or without pharmaceutically acceptable excipients;
b) optionally the core is layered with a cellulosic polymer;
c) desloratadine or its pharmaceutically acceptable salts are layered with or without an alkaline stabilizer; and
d) optionally an outer layer is a film coating with or without colorants.

In yet another embodiment of the invention:
a) a core comprises pseudoephedrine sulphate, hydroxypropyl methylcellulose, and ethyl cellulose, with or without other pharmaceutically acceptable excipients;
b) optionally the core is coated with a layer of hydroxypropyl methylcellulose polymer;
c) desloratadine or its pharmaceutically acceptable salts, a binder and one or more other pharmaceutically acceptable excipients are coated, as a layer, onto the core with or without an alkaline stabilizer; and
d) optionally an outer layer is a film coating comprising a film forming agent, plasticizer, colorants, and one or more other pharmaceutically acceptable excipients.

The therapeutically active ingredient is one or more compounds or their acceptable salts, solvates, enantiomers or mixtures such as but not limited to: sympathomimetic nasal decongestants like pseudoephedrine, phenylpropanolamine (PPA); non-steroidal anti-inflammatory drugs (NSAID) such as propionic acid derivatives like ibuprofen, naxproxen, flurbiprofen, fenoprofen, ketoprofen, suprofen, fenbufen and fluprofen; acetic acid derivatives like tolmetin sodium, zomepirac, sulindac and indomethacin; fenamic acid derivatives like mefenamic acid and meclofenamate sodium; biphenylcarboxylic acid derivatives like diflunisal and flufenisal; oxicams like piroxicam, sudoxicam and isoxicam; cox-2 inhibitors like celecoxib, rofecoxib, meloxicam, and nimesulide; bronchodilators like guaiphenesin, anti-tussitives such as dextromethorphan, codeine and pholcodeine; and opoid analgesics like naproxen and naltrexone.

In one of the embodiments, orally effective sympathomimetic nasal decongestants have been used in combination with desloratadine. Further, in an embodiment, pseudoephedrine or pharmaceutically acceptable salts, solvates, enantiomers or mixtures thereof have been used as an orally effective sympathomimetic nasal decongestant. The advantage of oral pseudoephedrine over topical nasal preparations is that it does not cause rebound congestion (“rhinitis medicamentosa”).

Stabilizers used in this invention include but are not limited to metal oxides such as magnesium oxide, amines such as meglumine, TRIS (tromethamine), ethanolamine, diethanolamine, triethanolamine, N-methyl-glucamine (meglumine), glucosamine, ethylenediamine, diethylamine, triethylamine, isopropylamine, diisopropylamine, and alkaline amino acids such as arginine, histidine, lysine and the like.

The w/w ratio of the stabilizing amount of stabilizer to the amount of desloratadine is in the range of about 4:1 to 1:4, or about 2:1 to 1:2.

An aspect of the present invention also includes a stable complex of an anti-allergic effective amount of desloratadine with porous copolymers of methacrylic acid crosslinked with divinylbenzene.

The ion exchange resin used is either a cation exchange resin or an anion exchange resin. Ion exchange resins useful in the practice of the present invention include but are not limited to anionic resins such as DUOLITE™ AP143/1083 (cholestyramine resin USP) and cationic resins such as AMBERLITE™ IRP-64 (a porous copolymer of methacrylic acid crosslinked with divinylbenzene), DUOLITE™ and AMBERLITE™ resins are available from Rohm and Haas Co.

In one of the embodiments, AMBERLITE™ IRP 64 is used as a complexing ion exchange resin to form a stable complex.

The w/w ratio of the stabilizing amount of ion exchange resin to the amount of desloratadine is in the range of about 4:1 to 1:4, or about 3:1 to 1:3.

In one of the embodiments of the present invention, the cellulosic polymers forming an inert barrier layer between a desloratadine layer and the hydrophilic core matrix comprising pseudoephedrine sulfate, comprises hydrophilic polymers such as but not limited to methylcellulose, ethyl cellulose, hydroxyethyl cellulose, hydroxypropyl cellulose, hydroxypropyl methyl cellulose, or mixtures thereof along with suitable processing aids such as plasticizers.

The above-mentioned polymer matrix containing pseudoephedrine sulfate further comprises binders, diluents or fillers, solvent systems for granulation and lubricants for compression.

An embodiment of the present invention provides for an inert polymeric barrier layer over the compressed matrix containing pseudoephedrine sulfate, the said inert polymeric barrier layer comprising one or more hydrophilic or hydrophobic polymer or mixtures thereof. An inert polymer of a barrier coating can be a hydrophilic cellulose polymer, such as hydroxypropyl methylcellulose, along with at least one excipient such as a suitable plasticizer, lubricant, antifoaming agent, solvent system, and optionally a colorant.

Another embodiment of the present invention provides for a second polymeric layer containing desloratadine onto the barrier layered polymeric matrix containing pseudoephedrine sulfate. The said second polymeric coat comprises desloratadine, one or more hydrophilic polymer or mixtures thereof, plasticizer, diluent, lubricant, antifoaming agent, chelating agent, solvent system and optionally, a colorant.

Further embodiments of the present invention provides for an optional third polymeric layer onto the second polymeric layer containing desloratadine, the said third outermost layer comprising one or more hydrophilic or hydrophobic polymer or mixtures thereof. Polymer of third outermost layer is hydrophilic cellulose, preferred being hydroxypropylmethyl cellulose, along with suitable plasticizer, lubricant, antifoaming agent, chelating agent, solvent system and optionally, a colorant.

In one of the embodiments, hydrophilic polymers of various grades are exemplified but are not limited to, celluloses such as carboxymethyl cellulose sodium, hydroxyethyl cellulose, hydroxypropyl methylcellulose (HPMC); homopolymers or copolymers of N-vinylpyrrolidone; vinyl and acrylic polymers; polyacrylic acid and the like; hydrophobic polymers such as celluloses like ethyl cellulose, low substituted hydroxypropyl cellulose (L-HPC), cellulose acetate, cellulose propionate (lower, medium or higher molecular weight), cellulose acetate propionate, cellulose acetate butyrate, cellulose acetate phthalate; polyalkyl methacrylates; polyalkyl acrylates; polyvinyl acetate (PVA); chitosan; stearic acid, gum arabic, crosslinked vinylpyrrolidone polymers; hydrogenated castor oil; and the like. Other classes of rate controlling substances or their mixtures in various ratios as required are also within the purview of this invention without limitation.

In the context of the present invention, during the preparation of the pharmaceutical compositions into finished dosage form, one or more pharmaceutically acceptable excipients may optionally be used which include but are not limited to: diluents such as microcrystalline cellulose (MCC), silicified MCC (e.g. Prosolv™ HD 90), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders such as acacia, guar gum, alginic acid, dextrin, maltodextrin, methylcellulose, ethyl cellulose, hydroxyethyl cellulose, hydroxypropyl cellulose (e.g. KLUCEL®), hydroxypropyl methylcellulose (e.g. METHOCEL®), carboxymethyl cellulose sodium, povidone (various grades of KOLLIDON®, PLASDONE®), starch and the like; chelating agents like edetate disodium USP; surfactants including anionic surfactants such as chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, sodium lauryl sulfate (SLS) and sodium dodecyl sulfate (SDS); cationic surfactants such as cetylpyridinium chloride monohydrate and hexadecyltrimethylammonium bromide; nonionic surfactants such as N-decanoyl-N-methylglucamine, octyl a-D-glucopyranoside, n-Dodecyl b-D-maltoside (DDM), polyoxyethylene sorbitan esters like polysorbates and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like; solvents that may be used in granulation or layering or coating are such as aqueous like water or alcoholic like ethanol, isopropanolol or hydro-alcoholic like a mixture of water with alcohol in any ratio or organic like acetone, methylene chloride, dichloromethane and the like.

Pharmaceutical compositions of the present invention may further include any one or more of pharmaceutically acceptable glidants, lubricants, opacifiers, colorants and other commonly used excipients.

In other embodiments, the pharmaceutical compositions of the present invention are filled into capsules (e.g. hard gelatin capsules). Pharmaceutical compositions to be filled into capsules can include pharmaceutically acceptable excipients, for example diluents such as cellulose, microcrystalline cellulose, mannitol, starch, and pregelatinized starch, and/or flow aids such as stearates.

Solid oral dosage forms of the present invention will be formulated to provide a unit dose of desloratadine of about 1 to 50 milligrams, or about 2.5 to 20 milligrams, or about 4 to 10 milligrams, about 2.5 milligrams, or about 5 milligrams.

Further, the oral dose range of pseudoephedrine or a salt thereof is about 100 to 450 milligrams, or about 200 to 300 milligrams, or about 200 to 250 milligrams, about 120 milligrams, or about 240 milligrams.

This invention thus provides compositions comprising an immediate release layer of an anti-allergic effective amount of desloratadine and a desloratadine-protective amount of a pharmaceutically acceptable carrier medium comprising substances that are basic in nature, or a stable complex of desloratadine with porous copolymers of methacrylic acid crosslinked with divinylbenzene. The sustained release core or layer comprises an effective amount pseudoephedrine or pharmaceutically acceptable salts, solvates, enantiomers or mixtures.

Thus desloratadine in combination with pseudoephedrine thus prepared may be a part of a pharmaceutical composition as tablets such as bilayered tablets, matrix-reservoir based systems, multi-unit particulate systems, tablet in tablet, capsules, sachets and the like. Such compositions could include other excipients as are required for the preparation of the compositions including but not limited to diluents, granulating agents, solvents, lubricants, wetting agents, disintegrating agents and the like. The procedures to convert such a blend into one of the compositions mentioned above are well known to a person skilled in the art of pharmaceutical formulations. This invention thus provides composition comprising an immediate release first layer of an anti-allergic effective amount of desloratadine and a desloratadine-protective amount of a pharmaceutically acceptable carrier medium comprising substances, which are basic in nature, or a stable complex of desloratadine with the porous copolymers of methacrylic acid crosslinked with divinylbenzene. The other layer or layers comprises an effective amount of one or more therapeutically active compounds or their pharmaceutically acceptable salts, solvates, enantiomers or mixtures in a sustained release or immediate release form.

The following examples will further describe certain specific aspects and embodiments of the invention in greater detail and are not intended to limit the scope of the invention.

### EXAMPLE 1

Method for the preparation of desloratadine polacrilin resinate.

1. One part of desloratadine was dispersed in water.
2. Citric acid was added to the above drug suspension until the pH of the suspension was about 6.5.
3. Three parts of polacrilin resin (Amberlite IRP 64) was added and stirred for 1-3 hours.
4. The dispersion obtained was filtered and dried at 60° C. to a loss on drying (LOD) <10% w/w.
5. The dried desloratadine polacrilin resinate was sifted through a 40 mesh ASTM sieve.

### EXAMPLE 2

Compositions for desloratadine—pseudoephedrine sulfate extended release tablets. (5 mg/240 mg)

| | |
|---|---|
| Ingredients | Kg per Batch |

Pseudoephedrine core

| | |
|---|---|
| Granulation | |
| Pseudoephedrine sulphate | 36 |
| Hydroxypropyl methylcellulose (K100M) | 48.5 |
| Ethyl cellulose (7 cps) | 12.2 |
| Povidone K30 | 5.7 |
| Water | 8 |
| Isopropyl alcohol | 24 |

Blending

| | |
|---|---|
| Colloidal silicon dioxide | 1.2 |
| Magnesium stearate | 1.5 |

Barrier layer

| | |
|---|---|
| Hydroxypropyl methylcellulose | 1.5 |
| Talc | 1.4 |
| Simethicone | 0.1 |
| Polyethylene glycol 8000 | 0.1 |
| Water | 28 |

Desloratadine layer

| | |
|---|---|
| Desloratadine | 1.1 |
| Polyacrylate dispersion 30% (Eudragit ™ NE 30D) | 4.3 |
| Talc | 1.5 |
| Simethicone | 0.1 |
| Polyethylene glycol 8000 | 0.4 |
| Water | 85 |

Manufacturing Process:
Pseudoephedrine Core:
1. Pseudoephedrine, hydroxypropyl methylcellulose, and ethyl cellulose, were sifted through a ASTM # 40 mesh, loaded into a rapid mixer granulator bowl and mixed for 10 minutes at high speed.
2. Povidone K30 was dispersed in an isopropyl alcohol and water mixture and stirred to form a clear solution.
3. The dry mix of step 1 was granulated using the povidone solution of step 2 in a rapid mixer granulator.
4. Granules were dried in a fluid bed drier at ambient temperature for 20 minutes and then the inlet air was heated to 75° C. and granules were dried until the loss on drying was less than 2% when measured using an infrared moisture balance at 105° C.
5. Dried granules were sifted through a 20 mesh ASTM sieve and sieve retains were milled using a comminuting mill with a 1 mm screen at medium speed with knives forward.
6. Colloidal silicon dioxide and magnesium stearate were sifted through a 60 mesh ASTM sieve.
7. Sifted and milled granules from step 5 and lubricants from step 6 were blended for 10 minutes in a double cone blender.
8. Lubricated granules were compressed into tablets using a rotary compression machine with 17.5×8.5 mm modified capsule shape punches and dies giving an average weight of 700 mg per tablet.
   Barrier Layer:
9. Hydroxypropyl methylcellulose was dispersed in water and stirred until a clear solution was obtained,
10. Polyethylene glycol 8000 and simethicone were added and stirred for 15 minutes and then talc was added and stirred for another 15 minutes.
11. Suspension was then milled through a colloid mill.
12. Tablets from step 8) were barrier layered using the suspension from step 11.
13. Tablets were dried for 120 minutes at 55° C.
   Desloratadine Layer:
14. Polyethylene glycol 8000 and simethicone were dispersed in water and stirred for 15 minutes, desloratadine was added and stirred for 15 minutes, then talc was added and stirred for another 15 minutes.
15. Dispersion was passed through a colloid mill for 10 minutes.
16. Eudragit NE 30 D was diluted with water and added to the dispersion of step 15 and stirred for 15 minutes.
17. Dispersion was filtered through a 60 mesh ASTM sieve.
18. Barrier layered tablets of step 13 were further layered with dispersion from step 17 maintaining the tablet bed temperature 30° C., inlet air temperature 45° C. to produce a desloratadine assay of not less than 98% of the desired amount.
19. Tablets were dried at 55° C. to get a loss on drying less than 6%.

### EXAMPLE 3

Compositions for desloratadine—pseudoephedrine sulfate extended release bilayer tablets. (5 mg/240 mg)

| | |
|---|---|
| Ingredients | mg per Unit |

Desloratadine layer

| | |
|---|---|
| Desloratadine polacrilin resinate of Example 1 | 25.3 |
| | (Equiv. to 5 mg |
| | desloratadine) |
| Mannitol (Pearlitol SD 200)* | 176.4 |
| Crospovidone | 10 |
| Lactose anhydrous | 25 |
| Colloidal silicon dioxide | 2 |
| Talc | 5 |
| Sodium stearyl fumarate | 6.3 |

Pseudoephedrine layer

| | |
|---|---|
| Pseudoephedrine sulfate | 240 |
| Kollidon SR** | 480 |
| Colloidal silicon dioxide | 5 |
| Magnesium stearate | 5 |

*Pearlitol SD 200 is manufactured by Roquette America Inc.
**Kollidon SR is manufactured by BASF Aktiengesell Schaft.

The desloratadine-pseudoephedrine extended release bilayer tablets (5 mg/240 mg) were prepared by direct compression as follows:
1. Desloratadine polacrilin resinate, crospovidone and mannitol were sifted together through a 40 mesh ASTM sieve.
2. Mannitol and lactose anhydrous were sifted through a 40 mesh ASTM sieve.
3. The sifted ingredients of step a) and b) were blended together in a double cone blender for 5 minutes
4. Talc, colloidal silicon dioxide and sodium stearyl fumarate were sifted together through a 60 mesh ASTM sieve and added to the double cone blender and blended for 5 minutes.
5. Pseudoephedrine, Kollidon SR, colloidal silicon dioxide were sifted together through a 40 mesh ASTM sieve and mixed uniformly.
6. Magnesium stearate was sifted through a 40 mesh ASTM sieve and blended with the pseudoephedrine blend of step e).
7. The desloratadine blend of step d) and pseudoephedrine blend of step f) were compressed into a bilayer tablet.
   Dissolution Conditions:

Apparatus: USP-II Stirring rpm: 50

Dissolution medium: 0.1 N HCl (1 hour) followed by pH 7.5 phosphate buffer

Volume of dissolution medium —1000 ml

Desloratadine - pseudoephedrine
sulfate extended release bilayer
tablets. (5 mg/240 mg) of Example 3

| | % Desloratadine | % Pseudoephedrine |
|---|---|---|
| Time (hours) | Released | Released |
| 1 | 95 | 26 |
| 4 | — | 48 |
| 8 | — | 69 |
| 12 | — | 82 |
| 16 | — | 89 |
| 20 | — | 92 |
| 24 | — | 95 |

### EXAMPLE 4

Composition for desloratadine-pseudoephedrine sulfate extended release tablets (5 mg/240 mg)

| | |
|---|---|
| Ingredients | mg per Unit |

Desloratadine layer

| | |
|---|---|
| Desloratadine polacrilin resinate of Example 1 | 25.3 |
| | (Equiv. to 5 mg |
| | desloratadine) |
| Mannitol (Pearlitol SD 200) | 176.4 |
| Crospovidone | 10 |
| Lactose anhydrous | 25 |
| Colloidal silicon dioxide | 2 |
| Talc | 5 |
| Sodium stearyl fumarate | 6.3 |

Pseudoephedrine layer

| | |
|---|---|
| Pseudoephedrine sulfate | 240 |
| Ethyl cellulose | 80 |
| Hypromellose K 100 M | 320 |
| Dibasic calcium phosphate | 108 |
| Povidone K 30 | 40 |
| Colloidal silicon dioxide | 8 |
| Magnesium stearate | 8 |

Manufacturing Process:
1. Desloratadine polacrilin resinate, crospovidone and mannitol were sifted together through #40 mesh sieve.
2. Mannitol and lactose anhydrous was sifted through # 40 mesh.
3. The sifted ingredients of step a) and b) were blended together in double cone blender for 5 minutes
4. Talc, colloidal silicon dioxide and sodium stearyl fumarate were sifted together through #60 mesh sieve and added to the double cone blender and blended for 5 minutes.
5. Pseudoephedrine sulfate, ethyl cellulose, hypromellose, dibasic calcium phosphate were sifted together through # 40 mesh sieve and were mixed uniformly.
6. The ingredients of step e) were granulated using a hydroalcoholic solution of providone K-30. The granules were dried at 60° C. and sifted through # 20 mesh sieve.
7. Colloidal silicon dioxide and magnesium stearate were sifted through # 40 mesh sieve and blended with the dried pseudoephedrine granules of step f)
8. The desloratadine blend of step d) and pseudoephedrine blend of step g) were compressed into a bilayer tablet.
   Dissolution Conditions:

Apparatus: USP-11 Stirring rpm: 50

Dissolution medium: 0.1 N HCl (1 hour) followed by pH 7.5 phosphate buffer

Volume of dissolution medium —1000 ml

Desloratadine - pseudoephedrine sulfate
extended release bilayer tablets
(5 mg/240 mg) of Example 4

| | % Desloratadine | % Pseudoephedrine |
|---|---|---|
| Time (hours) | released | released |
| 0 | 0 | 0 |
| 0.5 | 93 | 20 |
| 1 | 95 | 25 |
| 4 | — | 56 |
| 8 | — | 75 |
| 12 | — | 87 |
| 16 | — | 92 |
| 20 | — | 97 |

### EXAMPLE 5

Compositions for desloratadine—pseudoephedrine sulfate extended release tablets. (5 mg/240 mg)

Desloratadine layer composition with L-arginine (desloratadine: arginine=1:2 w/w)

| Ingredients | mg per Tablet |
|---|---|
| Desloratadine | 5 |
| L-arginine | 10 |
| Corn starch | 77.5 |
| Corn starch (for binder preparation) | 5 |
| Colloidal silicon dioxide | 1 |
| Polyethylene glycol (PEG) 6000 (fine grade) | 1.5 |

Manufacturing Process:
1. Desloratadine, stabilizer which is L-arginine, and corn starch were sifted through a 40 mesh ASTM sieve and mixed for 10 minutes in a rapid mixer granulator (RMG).
2. Starch paste was prepared using corn starch (ingredient 4) and water.
3. Ingredients of step a) were granulated using the starch paste of step b).
4. The granulated mass was dried in a fluid bed drier at a temperature of 65° C., until the moisture content of the granules was not more than 10%, as tested using an infrared moisture balance, and finally sifted through a 20 mesh ASTM sieve. The retained fraction was milled through a 1.5 mm sieve at medium speed knives forward and again sifted through the 20 mesh sieve.
5. Sifted colloidal silicon dioxide and PEG 6000 through a 60 mesh ASTM sieve and blended with the material of step d) in a double cone blender for 5 minutes.
6. The blend was compressed into tablets using 6.35 mm round tooling to a hardness of 3-8 kp. (kp is kilopond, a unit of force also called a kilogram of force, 1 kp=1 kgf).
7. The process for the preparation of a pseudoephedrine blend and compression into a bilayer tablet was followed as in Example 2.

### EXAMPLE 6

Composition of Desloratadine 5 mg and Pseudoephedrine sulfate 240 mg extended release tablets

| | |
|---|---|
| | Quantity per |
| Step | Batch |

| No. | Ingredient | Unit | Quantity |
|---|---|---|---|
| 1 | Granulation | | |
| | Pseudoephedrine sulfate | kg | 1 |
| | Hypromellose | kg | 1.4 |
| | Ethyl cellulose 7 cps | kg | 0.3 |
| | Povidone K 30 | kg | 0.2 |
| | Water | kg | 0.2 |
| | Isopropyl alcohol | kg | 0.7 |
| | Blending | | |
| | Colloidal silicon dioxide | kg | 0.03 |
| | Magnesium stearate | kg | 0.04 |
| 2 | Barrier layer | | |
| | Hypromellose 5 cps | g | 27.4 |
| | Talc | g | 27.4 |
| | Simethicone | g | 2.2 |
| | Polyethylene Glycol 8000 | g | 2.2 |
| | Water | g | 500 |
| 3 | Drug layer | | |
| | Desloratadine | g | 29.4 |
| | Polyacrylate dispersion 30 percent | g | 119.4 |
| | Talc | g | 41.2 |
| | Simethicone | g | 1.6 |
| | Polyethylene Glycol 8000 | g | 10.8 |
| | Water | g | 1600 |
| 4 | Top layer | g | |
| | Polyacrylate dispersion 30 percent | g | 70.2 |
| | Hypromellose 5 cps | g | 10.5 |
| | Talc | g | 29.2 |
| | Simethicone | g | 0.6 |
| | Polyethylene Glycol 8000 | g | 2.1 |
| | FD & C Blue # 2 Aluminium Lake | g | 0.2 |
| | Edetate Disodium | g | 0.5 |
| | Water | g | 570 |

Manufacturing Process:
1. Polyvidone was dispersed in the mixture of isopropyl alcohol and water.
2. Pseudoephedrine sulfate, hypromellose and ethyl cellulose were passed through ASTM # 40 mesh and mixed in rapid mixer granulator (RMG).
3. Mixture of step 2 was granulated using granulating solution of step 1 in a RMG for 25 minutes at impeller fast speed and chopper off.
4. Granules of step 3 were dried in fluidized bed drier for 20 minutes at inlet air temperature about 75° C., until loss on drying (LOD) was between 0.5% to 2.0% w/w, as measured at 105° C.
5. Granules of step 4 were milled using comminuting mill at medium speed and finally sifted through ASTM # 20 mesh sieve.
6. Granules of step 5 were lubricated with colloidal silicon dioxide and magnesium stearate by blending in double cone blender for 10 min.
7. Lubricated granules of step 6 were compressed in a rotary compression machine using 17.5 mm×8.5 mm modified capsule shaped punches and corresponding dies. Average tablet weight was 700 mg per tablet.
8. Barrier layer: Hypromellose, talc, polyethylene glycol were dispersed in water by stirring to get a uniform suspension. Core tablets of pseudoephedrine sulfate of step 7 were coated with this barrier layer suspension using coating pan until a weight built-up of 1.5% to 2.5% w/w was obtained.
9. Drug layer: Desloratadine and other excipients were dispersed in water by stirring to get a uniform suspension. Barrier layered tablets of step 8 were further layered with drug dispersion using coating pan until the assay for desloratadine was not less than 98%. Drug layered tablets were dried in coating pan until loss on drying (LOD) was less than 6.0% w/w, as measured at 105° C.
10. Top layer: All the excipients were dispersed in water by stirring to get a uniform suspension. Drug layered tablets of step 9 were further coated with top layer dispersion using coating pan to a weight built-up of 1.5% to 2.5% w/w. Top layered tablets were dried in coating pan until loss on drying (LOD) was less than 6.0% w/w, as measured at 105° C.
   Dissolution Conditions:

Apparatus: USP-II Stirring rpm: 50

Dissolution medium: 0.1 N HCl (1 hour) followed by pH 7.5 phosphate buffer

Volume of dissolution medium—1000 ml

Desloratadine - pseudoephedrine
sulfate extended release bilayer
tablets (5 mg/240 mg) of Example 6

| | % Desloratadine | % Pseudoephedrine |
|---|---|---|
| Time (hours) | Released | Released |
| 0.5 | 94 | — |
| 1 | 98 | 19 |
| 4 | — | 51 |
| 8 | — | 75 |
| 12 | — | 88 |
| 16 | — | 96 |
| 20 | — | 99 |
| 24 | — | 101 |

### EXAMPLE 7

Stability Data of desloratadine—pseudoephedrine sulfate extended release tablets (5 mg/240 mg) of Example 6.

Packaging for stability study: closed 60 cc HDPE bottle containing a silica gel canister and cotton.

| | | 1 month @ | 2 months @ | 3 months @ |
|---|---|---|---|---|
| | | 40° C. & | 40° C. & | 40° C. & |
| Parameter | Initial | 75% RH | 75% RH | 75% RH |
| Desloratadine (% | 102.1 | 104.6 | 103.2 | 99.5 |
| of theoretical) | | | | |
| Pseudoephedrine | 103.4 | 102 | 101.4 | 103.4 |
| sulphate (% of | | | | |
| theoretical) | | | | |
| N-Formyl | 0.08 | 0.19 | Nil | 0.3 |
| impurity (% w/w) | | | | |
| Dehydro impurity | Nil | Nil | 0.01 | 0.02 |
| (% w/w) | | | | |
| Total impurities | 0.21 | 0.37 | 0.48 | 1 |
| (% w/w) | | | | |

### EXAMPLE 8

Composition of Desloratadine 5 mg and Pseudoephedrine sulfate 240 mg Extended Release Tablets

Quantity per Batch

| Ingredient | Unit | Quantity |
|---|---|---|
| Granulation | | |
| Pseudoephedrine sulfate | kg | 36 |
| Hypromellose | kg | 48.5 |
| Ethyl cellulose 7 cps | kg | 12.2 |
| Povidone K 30 | kg | 5.7 |
| Water | kg | 8 |
| Isopropyl alcohol | kg | 24 |
| Blending | | |
| Colloidal silicon dioxide | kg | 1.2 |
| Magnesium stearate | kg | 1.5 |
| Barrier layer | | |
| Hypromellose 5 cps | kg | 1.5 |
| Talc | kg | 1.4 |
| Simethicone | kg | 0.1 |
| Polyethylene Glycol 8000 | kg | 0.1 |
| Water | kg | 28 |
| Drug layer | | |
| Desloratadine | kg | 1.1 |
| Eudragit NE 30 D | kg | 4.3 |
| Talc | kg | 1.5 |
| Simethicone | g | 60 |
| Polyethylene Glycol 8000 | kg | 0.4 |
| Water | kg | 8 |
| Top layer | | |
| Eudragit NE 30 D | kg | 4.2 |
| Hypromellose 5 cps | kg | 0.6 |
| (Methocel E5 Premium) | | |
| Talc | kg | 1.7 |
| Simethicone | g | 34 |
| Polyethylene Glycol 8000 | kg | 0.1 |
| FD & C Blue # 2 Aluminium Lake | g | 14.7 |
| Edetate Disodium | g | 14.7 |
| Water | kg | 30 |

Manufacturing Process: Similar to that Described in Example 6.

### EXAMPLES 9-10

Composition of Desloratadine 2.5 mg and Pseudoephedrine sulfate 120 mg extended release tablets with and without arginine.

| | | Quantity per Batch | | |
|---|---|---|---|---|
| | | | | Example |
| Step | Ingredient | Unit | Example 9 | 10 |
| I | Granulation | | | |
| | Pseudoephedrine sulfate | g | 1200 | 1200 |
| | Hypromellose | g | 1050 | 1050 |
| | Microcrystalline cellulose | g | 1000 | 1000 |
| | Povidone K 30 | g | 180 | 180 |
| | Water | g | 267 | 267 |
| | Isopropyl alcohol | g | 800 | 800 |
| | Blending | | | |
| | Colloidal silicon dioxide | g | 45 | 45 |
| | Magnesium stearate | g | 25 | 25 |
| II | Barrier layer | | | |
| | Hypromellose 5 cps | g | 48 | 48 |
| | Talc | g | 48 | 48 |
| | L-Arginine | g | — | 3 |
| | Polyethylene Glycol 8000 | g | 4.8 | 4.8 |
| | Water | g | 900 | 900 |
| III | Drug layer | | | |
| | Desloratadine | g | 35 | 35 |
| | Polyacrylate dispersion 30 per cent | g | 140 | 140 |
| | Hypromellose 5 cps | g | 21 | 21 |
| | Talc | g | 35.8 | 35.8 |
| | Simethicone | g | 2.0 | 2.0 |
| | L Arginine | g | — | 14 |
| | Polyethylene Glycol 8000 | g | 4.2 | 4.2 |
| | Water | g | 2000 | 2000 |
| IV | Top layer | | | |
| | Opadry Blue | g | 67.2 | 73.5 |
| | Water | g | 672 | 735 |

Manufacturing process: Similar to that described in Example 6.
Dissolution Conditions:

Apparatus: USP-II Stirring rpm: 50

Dissolution medium: 0.1 N HCl (1 hour) followed by pH 7.5 phosphate buffer

Volume of dissolution medium—1000 ml

Desloratadine -
pseudoephedrine
sulfate extended release bilayer
tablets (2.5 mg/120 mg) of Example 9

| | % Desloratadine | % Pseudoephedrine |
|---|---|---|
| Time (hours) | Released | Released |
| 0.5 | 96 | — |
| 1 | 96 | 19 |
| 4 | — | 75 |
| 8 | — | 94 |
| 12 | — | 100 |

We claim:

1. A composition comprising a sympathomimetic amine decongestant in a controlled release matrix tablet and one of:
a) a hydrophilic polymer barrier coating upon a controlled release matrix tablet, and a coating comprising desloratadine upon a hydrophilic polymer barrier coating;
b) a tablet layer comprising a desloratadine resinate complex, upon a controlled release matrix tablet; or
c) a tablet layer comprising desloratadine and an alkaline stabilizer upon a controlled release matrix tablet layer, or a coating comprising desloratadine and an alkaline stabilizer disposed upon a hydrophilic polymer barrier coating on a controlled release matrix tablet.

2. The composition of claim 1, wherein a sympathomimetic amine decongestant comprises pseudoephedrine or a salt thereof.

3. The composition of claim 1, wherein a hydrophilic polymer barrier coating comprises a cellulose derivative.

4. The composition of claim 1, wherein a hydrophilic polymer barrier coating comprises a hydroxypropyl methylcellulose.

5. The composition of claim 1, wherein an alkaline stabilizer comprises one or more of an amine, a metal oxide, and an alkaline amino acid.

6. The composition of claim 1, which is a bilayer tablet comprising a layer comprising a sympathomimetic amine decongestant in a controlled release matrix and a layer comprising a desloratadine resinate complex.

7. The composition of claim 1, which is a bilayer tablet comprising a layer comprising a sympathomimetic amine decongestant in a controlled release matrix and a layer comprising desloratadine and an alkaline stabilizer.

8. The composition of claim 1, which comprises a sympathomimetic amine decongestant in a controlled release matrix tablet, a hydrophilic polymer barrier coating upon a controlled release matrix tablet, and a coating comprising desloratadine upon a hydrophilic polymer barrier coating.

9. The composition of claim 8, wherein a hydrophilic polymer barrier coating comprises hydroxypropyl methylcellulose.

10. The composition of claim 1, which comprises a sympathomimetic amine decongestant in a controlled release matrix tablet, a hydrophilic polymer barrier coating upon a controlled release matrix tablet, and a coating comprising desloratadine and an alkaline stabilizer upon a hydrophilic polymer barrier coating.

11. A composition comprising a sympathomimetic amine decongestant in a controlled release matrix tablet having a hydrophilic polymer barrier coating, and a coating comprising desloratadine disposed over a hydrophilic coating.

12. The composition of claim 11, wherein a sympathomimetic amine decongestant comprises pseudoephedrine or a salt thereof.

13. The composition of claim 11, wherein a hydrophilic polymer barrier coating comprises a cellulose derivative.

14. The composition of claim 11, wherein a hydrophilic polymer barrier coating comprises a hydroxypropyl methylcellulose.

15. A composition comprising a tablet layer comprising a sympathomimetic amine decongestant in a controlled release matrix and a tablet layer comprising a desloratadine resinate complex.

16. The composition of claim 15, wherein a hydrophilic polymer barrier layer coating is interposed between a tablet layer comprising a sympathomimetic amine decongestant in a controlled release matrix and a tablet layer comprising a desloratadine resinate complex.

17. The composition of claim 15, wherein a hydrophilic polymer barrier coating comprises a hydroxypropyl methylcellulose.

18. A composition comprising a tablet layer comprising a sympathomimetic amine decongestant in a controlled release matrix and a tablet layer comprising desloratadine and an alkaline stabilizer.

19. The composition of claim 18, wherein wherein an alkaline stabilizer comprises one or more of an amine, a metal oxide, and an alkaline amino acid.

20. The composition of claim 18, wherein a hydrophilic polymer barrier layer coating is interposed between a tablet layer comprising a sympathomimetic amine decongestant in a controlled release matrix and a tablet layer comprising desloratadine and an alkaline stabilizer.

21. The composition of claim 18, wherein a hydrophilic polymer barrier coating comprises a hydroxypropyl methylcellulose.
